(19) United States
(12) Patent Application Publication
(10) Pub. No.: US 2011/0172461 A1
(43) Pub. Date: Jul. 14, 2011

(54) Polymer Recycling Methods Employing Catalytic Transfer Hydrogenation and Base Cleavage Reactions

(75) Inventor: Charles J. Rogers, Cincinnati, OH (US)

(73) Assignee: Rogers Family Living Revocable Trust

(21) Appl. No.: 12/674,714

(22) PCT Filed: Sep. 8, 2008
(86) PCT No.: PCT/US08/75537

§ 371 Date: Mar. 28, 2011

Related U.S. Application Data
(60) Provisional application No. 60/967,751, filed on Sep. 7, 2007.

(51) Int. Cl. C07C 27/02

(52) U.S. Cl. 562/483; 568/858

(57) ABSTRACT

Methods of recycling a post-consumer polymer material comprise depolymerizing the polymer material by heating the polymer material in the presence of a hydrogen donor material and a strong base compound, and optionally a catalyst, to effect catalytic transfer hydrogenation and base cleavage and produce intermediate and/or monomer products of molecular weights lower than that of the polymeric material. In a specific embodiment, the methods comprise recycling post-consumer polyethylene terephthalate. The methods comprise depolymerizing the polyethylene terephthalate by heating in the presence of a hydrogen donor material and a strong base compound, and optionally a catalyst, to effect catalytic transfer hydrogenation and base cleavage and produce terephthalic and/or naphthalic acid, or a salt thereof, and ethylene glycol.

### FIELD OF THE INVENTION

The present application claims priority under 35 U.S.C. §119 of U.S. Application No. 60/967,751 filed Sep. 7, 2007.

### FIELD OF THE INVENTION

The present invention is directed to methods of recycling polymers, for example, post consumer solid polymer material, using catalytic transfer hydrogenation and base cleavage reactions.

### BACKGROUND OF THE INVENTION

Catalytic transfer hydrogenation (CTH) research and development have been ongoing since the 1930's to develop safe and low cost methods to effect hydrogenation of organic compounds. Typical hydrogenation processes employ molecular hydrogen, hydrides and precious metal catalysts to promote the reduction of organic compounds.

Consumer use of plastics which are formed of polymer materials continues to increase and highlights the need for effective recycling. As an example, the use of plastic bottles for beverages is increasing while recycling of such bottles is relatively low. World wide it is estimated that more than 1.5 million tons of PET are collected per year although the European Trade Association has estimated that Europe alone PET collection will exceed one million tons by 2010. The 2007 U.S. Conference of Mayors called for research into the impact of discarded water and other beverage plastic bottles on municipal waste. The beverage industry itself is increasing efforts to promote the recycling and use of more recycled post-consumer plastics in the production of its soda, water, juice and tea bottles.

Virgin polyethylene terephthalate (PET) is used as a raw material to make bottles and other packaging materials for various products including soft drinks, alcoholic beverages, detergents, cosmetics, pharmaceutical products, and edible oils. Post consumer PET waste is often collected, crushed and pressed into bales which are offered for sale to recycling companies. Transparent colorless post-consumer PET attracts higher prices when compared to blue and green fractions as it contains reduced or no coloring pigment impurities. Recycling companies typically shred the collected PET into small fragments which often contain residues of the original content, paper labels, pigments and caps.

Various methods have been proposed and developed for the depolymerization of recycled PET to product oligomers and monomers. Goto et al, “Depolymerization of Polyethylene Terephthalate in Supercritical Methanol,” Journal of Physics: Condensed Matter, 14:11427-11430 (2002), disclose a batch process conducted at temperatures of 573-623 K, a pressure of 20 MPa and a reaction time of 20-120 minutes to convert PET to its monomers, i.e., dimethyl terephthalate (DMT) and ethylene glycol. The dimethyl terephthalate product must further be converted to terephthalic acid (TPA) before reuse to produce PET. Zope et al, “Studies of Degradation of Waste Poly(ethylene terephthalate) Using Autoclave Technique,” Chemical Engineering, The Institution of Engineers, India, 84:090309 (2003), depolymerize waste poly(ethylene terephthalate) using an autoclave technique wherein PET bottles were treated in a high pressure vessel with stirring at 250° C., a pressure of 16.596 Kg/cm, with and without lead acetate catalyst. The maximum depolymerization of PET with catalyst was 63.14%, using a processing time of two hours. U.S. Pat. No. 6,472,557 to Pell et al discloses a process to depolymerize PET to its component monomers, ethylene glycol and terephthalate (TPA). An exemplary process according to Pell et al comprised placing about 200 g of post consumer PET flakes in 400 g of methanol with a Zn catalyst (200 mg). The reactants were heated in an autoclave at a temperature 240° C. for two hours to convert PET to the DMT. The recovered DMT required heating for two additional 2 hours in an autoclave at 240° C. to produce TPA. Liu et al, “Hydrolytic Depolymerization of Polyterephthalate Under Microwave Irradiation,” Applied Polymer Science, 95(3):719-723, disclose hydrolytic depolymerization of PET using microwave irradiation. Their reaction was carried out in a sealed reaction vessel at a pressure of 20 bars and a temperature of 220° C., using a reaction time of 90-120 minutes and a weight ratio of water to PET of 10:1. Under these conditions, the PET was depolymerized completely.

Such conventional processes for depolymerizing post consumer PET typically involve glycenolysis or methanolysis and require high pressure, large molar amounts of expensive chemicals, and/or long reaction times to convert polyester feed stocks to TPA derivatives. Additional processing steps are often required to convert TPA derivatives such as DMT to TPA before use in the production of new products.

### SUMMARY OF THE INVENTION

Accordingly, it is an object of the present invention to provide methods for recycling polymers, for example, post consumer solid polymer material. It is another object of the invention to provide methods for recycling polymers using catalytic transfer hydrogenation and base cleavage reactions which effect depolymerization or decomposition of polymeric materials to lower molecular weight intermediates and monomers for use in the production of new products.

In one embodiment, the invention is directed to a method of recycling a post-consumer solid polymer material. The method comprises depolymerizing the polymer material by heating the polymer material in the presence of a hydrogen donor material and a strong base compound, and optionally a catalyst, to effect catalytic transfer hydrogenation and base cleavage and produce intermediate and/or monomer products of molecular weights lower than that of the polymeric material.

In another embodiment, the invention is directed to a method of recycling post-consumer polyethylene terephthalate. The method comprises depolymerizing the polyethylene terephthalate by heating in the presence of a hydrogen donor material and a strong base compound, and optionally a catalyst, to effect catalytic transfer hydrogenation and base cleavage and produce terephthalic and/or naphthalic acid, or a salt thereof, and ethylene glycol.

In a further embodiment, the invention is directed to a method of recycling post-consumer rubber. The method comprises depolymerizing the rubber by heating in the presence of a hydrogen donor material, a strong base compound and a catalyst to effect catalytic transfer hydrogenation and base cleavage and produce liquid and/or semi-liquid products of molecular weights lower than that of the rubber.

The present methods are advantageous in that the catalytic transfer hydrogenation and base cleavage avoid rigorous reaction conditions, typically produce intermediate and/or monomer products in relatively short times, often in a matter of minutes, and/or provide products that can be readily recovered for use in commerce. These and additional objects and advantages of various embodiments of the invention will be more readily understood and apparent in view of the following detailed description.

### DETAILED DESCRIPTION

The present invention is directed to methods for recycling of post-consumer polymer material and, more specifically, to methods wherein post-consumer solid polymer material is converted into reusable intermediate and/or monomer compound products. The methods of the present invention may be employed to recycle single or mixed polymeric materials and, depending on the starting polymer material(s), result in usable liquid and/or solid products and thus reduce landfill disposal of such polymer materials.

According to the present methods, the solid polymer material is depolymerized by heating the polymer material in the presence of a hydrogen donor material and an alkali metal or alkaline earth metal base compound, and optionally a catalyst, to effect catalytic transfer hydrogenation and base cleavage and produce intermediate and/or monomer products of molecular weights lower than that of the polymeric material. Intermediates may comprise dimers, trimers, or other oligomers of molecular weights small than the starting polymer material.

The polymer material which may be employed in the present methods may comprise any solid polymer material, including, but not limited to polyesters, polycarbonates, polystyrenes, natural or synthetic rubbers, bitumen, lignocellulose, polyolefins, or mixtures thereof, i.e., two or more of such polymers. The polymer material may comprise a homopolymer or a copolymer formed from at least one of the foregoing polymers. In a specific embodiment, the polymer material comprises a polyester polymer, and in a more specific embodiment, the polymer material comprises a polyethylene terephthalate having repeating units of terephthalic acid and ethylene glycol. As discussed above, polyethylene terephthalate is commonly used in plastic beverage bottles, so this specific embodiment of the invention provides an advantageous method for recycling such beverage bottles. In another embodiment, the polymer material comprises a polyolefin, and in a more specific embodiment, the polymer material comprises a polyethylene. The polyethylene may be high density (HDPE), low density (LDPE), linear low density (LLDPE), or other polyethylene polymer or homopolymer. As polyethylene is commonly employed in plastic containers, these embodiments of the present invention provide an advantageous method for recycling such containers. In yet another embodiment, the polymer material comprises a rubber, such as that of tires, and therefore the present invention provides an advantageous method for recycling used tires. The resulting liquid and semi-liquid intermediates may be used in the manufacture of new rubber products.

The methods of the present invention are conducted under treatment conditions which effect catalytic transfer hydrogenation followed by base cleavage. The catalytic transfer hydrogenation is initiated by the release of reactive hydrogen from a hydrogen donor material. The released reactive hydrogen breaks bonds in the polymer material. Water is produced by the base cleavage reaction. A portion of this water may be consumed in the formation of the intermediates and monomers, and any remaining water may be removed from the reaction system if desired. For example, in the method employing polyethylene terephthalate (PET) as the polymer material, the reactive hydrogen breaks ester bonds between terephthalic acid and ethylene glycol and water is produced. An example of the conversion of PET to disodium terephthalate (Na₂TPA) and/or monosodium terephthalate (NaTPA) and ethylene glycol is as follows:

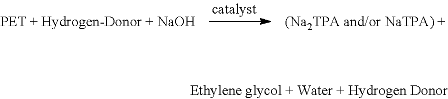

One skilled in the art will appreciate that sodium naphthalate may also be produced in this reaction, depending on the polyester composition. Theoretically, two moles of ethylene glycol are produced for each mole of acid which is produced. Typically, the products of such a reaction will comprise about two thirds by weight of the acid or acid salt and one third by weight ethylene glycol.

Suitable hydrogen donor materials include, but are not limited to, C₈-C₃₀ hydrocarbons, liquid hydrocarbon polymers, glycols, for example, ethylene glycol, oils, for example hydrocarbon oils, or a mixture thereof. For example, in the depolymerization of polyethylene terephthalate, ethylene glycol may be employed as a hydrogen donor material. Waste sources of ethylene glycol from car cooling systems, deicing of airplanes, and the like, can be collected and reused after cleanup as a low cost reaction medium if desired. As ethylene glycol is also produced in the depolymerization of polyethylene terephthalate, the use of the ethylene glycol as a hydrogen donor material provides an economic advantage to the process. Additionally, ethylene glycol produced in excess of that suitable for use as the hydrogen donor material may be employed for other commodity and industrial uses. As another example, hydrocarbon oil may be used in the depolymerization of rubber. The hydrogen donor material has a high boiling point to maintain the material in liquid form during the reaction. Thus, the hydrogen donor material conveniently provides a reaction medium. The hydrogen donor material releases reactive hydrogen to initiate the depolymerization reaction. The strong base compound and, optionally, the catalyst, promote the release of reactive hydrogen from the hydrogen donor material. As the reactive hydrogen is not consumed, the hydrogen donor material may conveniently be reused. The hydrogen donor material may be used in an amount sufficient to provide reactive hydrogen in an amount to initiate the reaction. In a specific embodiment, the hydrogen donor material is provided in an amount sufficient to provide a reaction medium. In a more specific, the hydrogen donor material comprises from about 20 to about 80% of the reaction volume, or, more specifically, from about 30 to about 60% by volume of the reaction volume.

The strong base compound has a pH of at least about 9, more preferably at least about 10. In a specific embodiment, the strong base compound comprises an alkali metal or alkaline earth metal base compound and in a more specific embodiment comprises one or more of hydroxides. In a specific embodiment, the base compound comprises an alkali metal hydroxide, and in yet a more specific embodiment, the base compound comprises sodium hydroxide. The base compound is employed in the reaction mixture in an amount sufficient to promote the release of reactive hydrogen from the hydrogen donor material for the depolymerization reaction and to participate in the base cleavage reaction. In a further embodiment, the base compound is employed in the reaction in a stoichiometric amount for producing salt products, if desired. For example, in the depolymerization of PTE, the base compound NaOH can be employed in an amount to provide two moles of sodium for each produced mole of terephthalic acid.

As noted, the depolymerization reaction may optionally employ a catalyst. Suitable catalysts comprise, but are not limited to, saturated and unsaturated fatty acids, alcohols, carbon, or a mixture thereof, i.e., a mixture of two or more of the foregoing. The catalyst is employed in an amount sufficient to promote production of the reactive hydrogen from the hydrogen donor material and may suitably be employed in the reaction medium in an amount of 0.1 to 10% by volume of the depolymerization reaction volume. Many depolymerization reactions will proceed in the absence of the catalyst, although the use of a catalyst may reduce overall reaction times.

The depolymerization reaction may be conducted in the presence of an added reaction medium, in addition to the hydrogen donor material, if desired.

The temperature of the heating step may be varied, dependent on the starting material, hydrogen donor material, catalyst, if employed, and the like. Typically, however, the present methods may be readily conducted at a temperature above about 100° C. In a specific embodiment, the heating is conducted at a temperature in a range of from about 120° C. to about 450° C., or, in more specific embodiments, at a temperature in a range of from about 120° C. to about 350° C. or in a range of from about 200° C. to about 450° C. In further embodiments, the heating may be conducted at a temperature in a range of from about 120° C. to about 150° C. In a more specific embodiment, wherein the starting material comprises polyethylene terephthalate, the heating may be conducted at a temperature in a range of from about 120° C. to about 150° C. or from about 120° C. to about 195° C. The heating may be conducted in a pressurized or non-pressurized vessel. In one embodiment, the heating step is conducted in a reactor provided with a nitrogen-containing head space. Typically, a nitrogen head space will be employed in higher temperature heating steps but it may also be used with lower temperature heating steps if desired. The heating step is typically conducted for a period of time of less than several, i.e., about three, hours. In one embodiment, the heating step is conducted for less than about two hours, and, in another embodiment, the heating step is conducted for about 20 to about 60 minutes. As suitable, water may be removed from the reactor as the depolymerization reaction proceeds. In a specific embodiment, complete depolymerization of polyester material such as PET to TPA or a salt thereof is achieved by heating in a method according to the present invention for about 20 to about 60 minutes at temperatures ranging from about 120-350° C., or, more specifically, ranging from about 120° C. to less than about 195° C.

The depolymerization reaction will result in intermediate and/or monomer products in liquid or solid form having molecular weights lower than that of the polymer material. The base compound is available for reaction with one or more products and may assist in precipitating the same to facilitate removal from the reaction medium. Any precipitated intermediate or monomer products may conveniently be removed by filtration, centrifugation, or the like. In the depolymerization of polyethylene terephthalate, the resulting sodium terephthalate may be converted to terephthalic acid form by discharging the reaction medium into a mineral acid solution with stirring. The terephthalic acid immediately precipitates as it is an insoluble acid and may be recovered by filtration or centrifugation.

After the depolymerization reactions are completed, the reaction medium is cooled and the products are recovered from the reaction medium by filtration or centrifugation, or other separation processes known in the art. In specific embodiments, TPA and other monomers may be recovered in amounts of greater than 50%, more specifically, greater than 75%, and, in certain instances, of 95-99% yields. Importantly, contaminants and toxic pollutants which may have been present in the polymer material feed stock, for example, residual content, labels, plastic bottle caps and the like, may be destroyed during the depolymerization reaction. The high yields and high quality of the recovered monomers and oligomers allow use of the resulting products in the production of new virgin products.

The following Examples demonstrate specific embodiments of the invention, but should not be construed as limiting the scope of the invention.

### EXAMPLE 1

One specific embodiment of the method of the present invention comprises placing 300 ml of hydrogen donor, 200 grams of PET (cut into ¼ to ½ inch sized pieces), 40 grams sodium hydroxide, and polybutadiene catalyst (1.0% of the reaction medium volume) into a 1-liter vessel. The mixture is heated to a temperature in the range of about 110-220° C. and maintained at a temperature with this range, with stirring, for 20 to 30 minutes. The contents of the vessel is cooled and centrifuged at 5000-10,000 rpm to recovery the products comprising TPA and the sodium salt of TPA. The depolymerization of PET is achieved in the presence or the absence of the catalyst, although the catalyzed depolymerization reactions typically proceed more quickly than non-catalyzed reactions.

### EXAMPLE 2

Another specific embodiment of the method of the present invention comprises placing 300 grams of PET (cut into ¼ to ½ inch sized pieces), 400 ml ethylene glycol, 150 grams sodium hydroxide, and hexabutadiene catalyst (1-5% of volume of the reaction medium) into a 1000 ml round bottom three neck flask. A temperature probe, stirrer and Dean-Stark trap are placed in position. The contents of the flask are heated with stirring to a temperature of about 150° C. and then above, but are maintained below about 195° C. At about 120-130° C., depolymerization commences. The depolymerization rate is accelerated at a temperature of about 150° C. and above. The depolymerization however can be completed at the lower temperature range of 120-130° C. The reaction is completed within about 15 to 30 minutes. During the heating, water is distilled from the reaction medium and collected in the Dean-Stark trap. When stirring of the reaction medium ceases, the terephthalate product readily settles to the bottom of the reaction flask and is easily removed. The initial ethylene glycol and the ethylene glycol produced from the reaction are removed by decantation, centrifugation or filtration for maximum recovery of added and produced ethylene glycol. The depolymerization of PET is achieved in the presence of the catalyst as described, or in the absence of the catalyst, although the catalyst may improve the depolymerization reaction rate depending on the base concentration.

### EXAMPLE 3

Another specific embodiment of the method of the present invention comprises recycling post consumer PET bottles wherein the PET contains non-PET fibers to help retain its form. To determine a suitable amount of sodium hydroxide for use in this method, it is assumed that 70% by weight of the product subject to recycling is PET polymer and the remaining materials are non-PET. Accordingly, if 1000 lbs of product is to be processed, it is assumed that 700 lbs is PET. As the PET comprises two moles of ethylene glycol (MW of 124 (62×2)) combined with each mole of terephthalic acid (MW 166), the total weight of a diethylene glycol terephthalic unit is 288. The number of mole pounds in 700 lb of PET is therefore 2.4 mole pounds (700 lbs/288 lbs/mole lb). As 2.4 mole lbs of NaOH (MW 40) will be required to produce the monosodium salt or 4.8 mole lbs of NaOH will be required to produce the disodium salt, 96 lb of NaOH should be added to produce the monosodium salt or 192 lbs of NaOH should be added to produce the disodium salt of terephthalic acid. While it would appear more cost effective to produce dimers or oligomers since less sodium hydroxide is required, sodium hydroxide is a low cost chemical and therefore the cost for this material is not a significant factor.

### EXAMPLE 4

Another specific embodiment of the method of the present invention comprises converting tire rubber to a liquid or semi-liquid product. The method comprises combining 500 ml of hydrogen donor comprising a hydrocarbon oil in a reactor with 300 grams of rubber, cut to pieces of an inch or less in size, sodium hydroxide, and a carbon catalyst. The reaction mixture is heated at a temperature of 150-240° C. for about 20 to 60 minutes. The carbon catalyst is very effective in rubber depolymerization, specifically devulcanization, into a liquid or semi-liquid products. The resulting products may be used in manufacture of new rubber products.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments described in the specification and/or the examples. As one of ordinary skill in the art will readily appreciate from the disclosure of the present invention, additional embodiments, presently existing or later to be developed, that perform substantially the same function or achieve substantially the same result as the corresponding embodiments described herein may be utilized according to the present invention. Accordingly, the appended claims are intended to include within their scope such additional embodiments.

1. A method of recycling a post-consumer solid polymer material, comprising depolymerizing the polymer material by heating the polymer material in the presence of a hydrogen donor material and a strong base compound, and optionally a catalyst, to effect catalytic transfer hydrogenation and base cleavage and produce intermediate and/or monomer products of molecular weights lower than that of the polymeric material.

2. The method of claim 1 wherein the polymer material comprises polyester, polycarbonate, polystyrene, natural or synthetic rubber, bitumen, lignocellulose, polyolefin, or a mixture thereof.

3. The method of claim 1, wherein the heating is conducted at a temperature above about 100° C.

4. The method of claim 1, wherein the heating is conducted at a temperature in a range of from about 120° C. to about 450° C.

5. The method of claim 1, wherein the heating is conducted at a temperature in a range of from about 120° C. to about 350° C.

6. The method of claim 1, wherein the heating is conducted at a temperature in a range of from about 120° C. to about 150° C.

7. The method of claim 1, wherein the base compound comprises alkali metal hydroxide.

8. The method of claim 1, wherein the hydrogen donor comprises a C₈-C₃₀ hydrocarbon, a glycol, a liquid hydrocarbon polymer, oil, or a mixture thereof.

9. The method of claim 1, wherein a catalyst is employed in the heating step.

10. The method of claim 9, wherein the catalyst comprises a saturated or unsaturated fatty acid, an alcohol, carbon, or a mixture thereof.

11. The method of claim 9, wherein the catalyst is employed in an amount of 0.1 to 10% of the volume of the depolymerization reaction.

12. The method of claim 1, wherein water is removed during the depolymerization step.

13. The method of claim 1, wherein the depolymerization step is conducted in a non-pressurized system.

14. The method of claim 1, wherein the depolymerization step is conducted in a reactor provided with a nitrogen-containing head space.

15. The method of claim 1, wherein the intermediate and/or monomer products are removed by centrifugation or filtration from a reaction medium in which the depolymerization is conducted.

16. A method of recycling post-consumer polyethylene terephthalate, comprising depolymerizing the polyethylene terephthalate by heating in the presence of a hydrogen donor material and a strong base compound, and optionally a catalyst, to effect catalytic transfer hydrogenation and base cleavage and produce terephthalic and/or naphthalic acid, or a salt thereof, and ethylene glycol.

17. The method of claim 16, wherein sodium terephthalate and/or sodium naphthalate are produced in the depolymerization step and the method further comprises separating the sodium terephthalate and/or sodium naphthalate as a precipitate from ethylene glycol.

18. The method of claim 16, wherein the hydrogen donor material comprises ethylene glycol.

19. The method of claim 16, wherein the heating is conducted at a temperature in a range of from about 120° C. to about 450° C.

20. The method of claim 6, wherein the heating is conducted at a temperature in a range of from about 120° C. to about 150° C.

21. A method of recycling post-consumer rubber, comprising depolymerizing the rubber by heating in the presence of a hydrogen donor compound, a strong base compound, and a catalyst to effect catalytic transfer hydrogenation and base cleavage and produce liquid and/or semi-liquid products of molecular weights lower than that of the rubber.

22. A method of recycling a post-consumer solid polymer material, comprising depolymerizing the polymer material by heating the polymer material in the presence of a hydrogen donor material and a strong base compound, and optionally a catalyst, to effect catalytic transfer hydrogenation and base cleavage and produce intermediate and/or monomer products of molecular weights lower than that of the polymeric material, wherein the intermediate and/or monomer products react with the strong base to form salts and wherein contaminants and/or toxic pollutants in a feedstock of the polymer material are destroyed.
